(19) United States
(12) Patent Application Publication
(10) Pub. No.: US 2004/0265262 A1
(43) Pub. Date: Dec. 30, 2004

(54) Compound derived from polyguanidine and use of such a compound for protection and/or care of keratinous fibres

(76) Inventors: Michel Philippe, Wissous (FR); Alain Campos, Mitry-Mory (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/484,320

(22) PCT Filed: Jul. 9, 2002
(86) PCT No.: PCT/FR02/02395

(30) Foreign Application Priority Data
Jul. 18, 2001 (FR) 01/09633

(51) Int. Cl. A61K 7/06; A61K 7/11

(52) U.S. Cl. 424/70.17

(57) ABSTRACT

The invention concerns the cosmetic use of polyguanidines or of their physiologically acceptable salts for or in a composition for the protection and/or care of keratinous fibres, as well as cosmetic compositions containing polyguanidines. The invention also concerns a novel family of polyguanidines.

The present invention relates to the cosmetic use of polyguanidines or physiologically acceptable salts thereof for protecting and/or caring for keratin fibers, and also to compositions containing polyguanidines.

The invention also relates to a novel family of polyguanidines.

Polyalkyleneguanidines are copolymers obtained by condensation of guanidine hydrochloride with an alkylenediamine. Such compounds and the preparations thereof from hexamethylenediamine have been known since 1975 by Zh. Prikl. Khim. (Leningrad) (1975), 48 (8), 1833-6, from Gembitskii, P. A. et al.

Polyalkyleneguanidines are known for their antibacterial activity, and this use is especially described in patents RU 2 143 905 and SU 1 687 261. More recently, the use of polyalkyleneguanidines for skin care has been described. Thus, patents SU 1 803 099 and RU 2 106 859 describe the cicatrizing and emollient properties of these compounds.

Patent application SU 857 257 describes the highly detergent properties of compositions based on polyhexamethyleneguanidines.

Various processes are known for synthesizing these polyalkyleneguanidine derivatives.

Patent applications EP 0 439 698, WO 99/54291 or RU 2 052 453 present alternatives to the processes for synthesizing polyalkyleneguanidines described in the publication by Gembitskii et al. cited above.

Advantageously and surprisingly, the Applicant has discovered that compounds derived from poly-alkyleneguanidines of formula (I) that will be defined below can be used in cosmetics to care for and/or protect keratin fibers.

The Applicant has thus shown especially that compositions containing such compounds derived from polyguanidines make it possible to maintain or even restore the mechanical properties of the hair. The hair remains soft, is not brittle and has an appearance that satisfies the esthetic requirements in terms of sheen and silkiness. These compounds derived from polyguanidines are excellent hair conditioners, allowing the fibers to acquire a soft and silky feel.

These properties have particularly been observed on sensitized hair, such as hair that has, in the past, been regularly subjected to oxidation dyeing or permanent-waving operations.

In particular, the compositions according to the invention may be used as shampoos or conditioners. In all these applications, the compositions according to the invention allow treated keratin fibers to obtain a very soft feel and allow the disentangling to be improved. This improved conditioning effect was verified in the presence of an aqueous base and also in the presence of a surfactant base.

In the presence of an aqueous and/or surfactant base, improved disentangling and a softer, smoother and less coarse feel were observed.

In addition, these compounds derived from polyguanidines have the advantage of being more environmentally friendly than the polymers of the prior art used in cosmetics.

The compounds derived from polyguanidine that may be used in the context of the present invention correspond to formula (I) below:

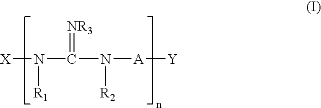

in which:

X, R₁, R₂ and R₃, independently of each other, are chosen from the group formed by a hydrogen atom, a hydroxyl radical and a linear or branched, saturated or unsaturated, optionally hydroxylated C₁ to C₁₆ and preferably C₁ to C₈ alkyl radical,

Y denotes a radical NHR, in which R is chosen from the group formed by a hydrogen atom, a hydroxyl radical and a linear or branched, saturated or unsaturated, optionally hydroxylated C₁ to C₁₆ and preferably C₁ to C₈ alkyl radical,

A denotes a linear or branched, saturated or unsaturated, C₁ to C₁₆ and preferably C₁ to C₁₂ alkylene radical optionally substituted with at least one radical chosen from a hydroxyl, carboxyl (—COOH) or carboxylate radical and a halogen, said alkylene radical possibly containing at least one amine (—NH—), ether (—O—), thioether (—S—), ester (—(CO)O— or —O(CO)—), amide (—CONR— or —NRCO— in which R is hydrogen or a C₁ to C₈ alkyl radical), carbamate (—NH(CO)O— or —O(CO)NH—) or urea (—NH(CO)NH—) function, a C₆ aryl or C₃ to C₈ cyclanyl ring optionally substituted with a C₁—C₈ alkyl, hydroxyl or halogen; or A denotes one or more C₆ or C₇ aryl or C₅ to C₇ cyclanyl rings substituted with a C₁—C₈ alkyl, a hydroxyl group or a halogen or are unsubstituted; or A denotes a C₁₀ to C₁₄ polyaryl or C₆ to C₁₀ polycyclanyl chain, which may be interrupted with at least one C₁ to C₈ alkylene radical, an amine (—NH—), amide (—CONR— or NRCO in which R is a C₁ to C₈ alkyl radical), ether (—O—), thioether (—S—), a hydrogen atom, carbamate (—NH(CO)O— or —O(CO)NH—) or urea (—NH(CO)NH—) function;

n is an integer between 2 and 5 000 and preferably between 2 and 200, and the physiologically acceptable salts thereof.

For the purposes of the present patent application, the term “carboxylate” means an addition salt of a carboxylic acid with a base chosen especially from sodium hydroxide, potassium hydroxide, aqueous ammonia, amines or alkanolamines or alternatively an ampholytic internal salt with a guanidinium group of the chain.

For the purposes of the present patent application, the expression “C₆ aryl ring” means a benzene nucleus, this nucleus possibly being substituted with one or two C₁ or C₈ alkyl radicals, OH or halogen.

For the purposes of the present patent application, the expression “C₁₀ to C₁₄ polyaryl chain” means a chain containing 2 or 3 aromatic nuclei, each optionally being substituted with one or two C₁ to C₈ alkyl radicals, OH or halogen.

Preferably, they will more particularly be derivatives of formula (I) defined above, in which A denotes a linear or branched, saturated or unsaturated C₁ to C₆ alkylene radical, optionally substituted with at least one radical chosen from hydroxyl, carboxyl and carboxylate radicals or halogens (fluorine, chlorine, bromine or iodine).

As compounds derived from polyguanidines, either homopolymers or heteropolymers may be used.

For the purposes of the present patent application, the term “homopolymer” means a polymer chain consisting of (—N(R₁)—C(NR₃)—N(R₂)-A) units, in which R₁, R₂, R₃ and A are always identical, and the term “heteropolymer” means a polymer for which at least one of the units R₁, R₂, R₃ and A is different from the others.

Even more preferably, the compounds used in the compositions for protecting and/or caring for keratin fibers, within the meaning of the present invention, are polyethyleneguanidinium, polytetramethylene-guanidinium and polyhexamethyleneguanidinium salts and more particularly the halides (fluoride, chloride and bromide), the carboxylates (gluconates, acetates, lactates, oleates and laurates) or an ampholytic internal salt with a guanidinium group of the chain.

The compounds derived from polyguanidines that are particularly preferred are polyethyleneguanidinium oleate, polyethyleneguanidinium chloride, polytetra-methyleneguanidinium oleate, polytetramethylene-guanidinium acetate and polytetramethyleneguanidinium chloride.

The polyguanidine derivatives that may be used in the context of the present invention may be prepared by carrying out any preparation process known in the prior art. In particular, the compounds derived from polyguanidine according to the present invention are prepared by mixing an alkylenediamine and a guanidine salt, for example guanidine hydrochloride, in an approximately equimolar ratio, followed by heating this mixture to a temperature of between 120 and 150° C. for a period of between 4 and 10 hours. The alkylenediamine and the guanidine salt may be mixed together in bulk or in the presence of a solvent, which will preferably be polyethylene glycol (PEG).

PEG has the advantage of being a good solvent for the reagents: alkylenediamine and guanidine salt; on the other hand, the compound derived from polyguanidine obtained after the reaction is immiscible with PEG. This process thus makes it possible to recover the compound derived from polyguanidine directly and also to recover the PEG, which may be reused as solvent, and also the excess reagents or reagents that have not had time to react.

According to another variant of the process for preparing the compounds derived from polyguanidines according to the invention, the heating step may be carried out in two stages: a first step of heating at a temperature of between 80° C. and 120° C., for a period of between 2 and 5 hours, optionally followed by a second step of heating at a temperature of between 120° C. and 150° C., for a period of between 5 and 11 hours.

The process of Gembitskii et al., cited above, and also the processes described in patent applications WO 99/54291 and EP 439 698 may be used to prepare the compounds derived from polyguanidines according to the present invention.

Among the polyguanidine derivatives that may be used in the context of the present invention, a certain number are novel and constitute another subject of the invention.

They are compounds of formula (II) below:

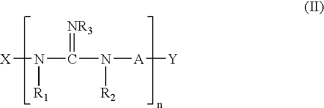

in which X, R₁, R₂, R₃, Y, n and A have the same meanings indicated above in formula (I) above, with the proviso that A does not denote an unsubstituted linear C₁-C₁₆ alkylene radical; in addition, these compounds may be prepared according to the processes mentioned above.

Preferably, the compounds of formula (II) are such that A denotes a linear or branched, saturated or unsaturated C₁ to C₁₂ alkylene radical, substituted with at least one radical chosen from a hydroxyl, carboxyl or carboxylate radical or a halogen.

These compounds of formula (II) may be used in compositions, especially cosmetic compositions, for protecting and/or caring for keratin fibers and even more preferably the hair.

As indicated above, the polyguanidine derivatives that may be used in the context of the present invention have advantageous cosmetic properties that allow them to be used in the preparation of cosmetic compositions.

A subject of the present invention is also the use of at least one polyguanidine derivative of formula (I) in cosmetic compositions for protecting and/or caring for keratin fibers, in particular the hair.

These cosmetic compositions containing at least one compound derived from polyguanidine generally comprise at least one adjuvant usually used in cosmetic compositions.

These cosmetic compositions may be, for example, in the form of aqueous, alcoholic or aqueous-alcoholic solutions (the alcohol especially being a lower alcohol such as ethanol or isopropanol), or in the form of creams, gels or emulsions, or alternatively in the form of aerosols also containing a propellant, for instance nitrogen, nitrous oxide or hydrocarbons (butane, propane, etc.), or sprays.

The adjuvants generally present in the cosmetic compositions of the invention are, for example, fragrances, preserving agents, sequestering agents, thickeners and emulsifiers.

It should be noted that the cosmetic compositions according to the invention are either ready-to-use compositions or concentrates that need to be diluted before use. The cosmetic compositions of the invention are thus not limited to a particular concentration range of compounds derived from polyguanidines.

Generally, in the cosmetic compositions of the invention, the concentration of compounds derived from polyguanidines is between 0.001% and 25% by weight and preferably between 0.1% and 10% by weight relative to the total weight of the composition.

The compounds derived from polyguanidines especially have advantageous cosmetic properties when they are applied to the hair.

Thus, when they are applied to the hair, either alone or with other active substances during a treatment, they substantially improve the qualities of the hair.

For example, they promote the treatment and facilitate the disentangling of wet hair. Even at high concentration, they do not give the wet hair a sticky feel.

In contrast with the usual cationic agents, they do not make dry hair lank, and thus facilitate bouffant hairstyles. They give dry hair liveliness qualities and a shiny appearance.

They contribute efficiently toward eliminating the defects of hair sensitized by treatments such as bleaching, permanent-waving or dyeing operations. Specifically, it is known that sensitized hair is often dry, dull and coarse, and difficult to disentangle and style.

They are in particular of great advantage when they are used as pretreating agents, especially before shampooing with an anionic and/or nonionic shampoo or before oxidation dyeing, itself followed by shampooing with an anionic and/or nonionic shampoo. The hair is then particularly easy to disentangle and has a very soft feel.

They may also be used as pretreating agents in other hair treatment operations, for example permanent-waving treatments.

The compounds derived from polyguanidines may be present in the haircare cosmetic compositions according to the invention either as additive or as main active ingredient, in hairsetting lotions, treatment lotions, styling creams or gels, restructuring lotions, anti-seborrheic treatment lotions, or hair lacquers, and also in shampoos and conditioners. The compounds derived from polyguanidines may be used in combination with any other cosmetically acceptable active agent in particular, in compositions conventionally used in haircare.

For example, to obtain a cream, an aqueous phase containing in solution the compound derived from polyguanidines and optionally other ingredients or adjuvants, and an oily phase, may be emulsified.

The oily phase may consist of various products such as liquid paraffin, liquid petroleum jelly, sweet almond oil, avocado oil, olive oil, fatty acid esters, for instance glyceryl monostearate, ethyl palmitate or isopropyl palmitate, or alkyl myristates, such as propyl myristate, butyl myristate or cetyl myristate. Fatty alcohols such as cetyl alcohol, or waxes, for instance beeswax, may also be added.

A subject of the invention is also a cosmetic process for protecting keratin fibers, characterized in that at least one compound derived from polyguanidines of formula (I) is applied to the keratin fibers.

### EXAMPLES

I. Synthesis of Polylysylimmonium Chloride

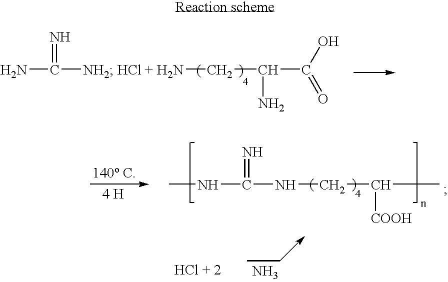

Procedure

40 mmol of guanidinium chloride (3.82 g) and 40 mmol of lysine (5.84 g) are introduced into a round-bottomed flask.

The mixture is heated to 90° C. Once the medium has become fluid, stirring is started and maintained at this temperature for 30 minutes, and the mixture is then brought to 140° C. Considerable evolution of ammonia and thickening of the medium are observed. After 4 hours, the evolution of ammonia has ceased.

The heating is then stopped.

After cooling, a slightly fluorescent dark yellow resin is obtained.

II. Test of Rigidity of the Hair

The test of tensile dynamic mechanical characterization on dry hair and in water makes it possible to evaluate the intrinsic viscoelastic properties of the hair under a controlled atmosphere and in water, and makes it possible to determine the mechanical properties of the hair in the course of standard living conditions. When the hair is degraded, the decrease in the rigidity of the hair, especially in water, is greater than for undegraded hair.

Protocol

Dyed and permanent-waved hair is mounted in sets of three onto dynamic characterization terminals. The tests are performed under the following conditions:

specimen length: 20 mm

static force: 0.5 N

dynamic elongation: 20 μm

stress frequency: 15.6 Hz

“dry” characterization (T=20° C.; RH=45%): 2 minutes

“immersion” characterization (water: room temperature): 3 minutes

Measurement Protocol

A first series of measurements is performed before treatment.

Next, for each product, five tests are performed, each time on a 1 g lock. These locks are treated using a pretreatment solution before oxidation dyeing for 30 minutes. Solutions C1 and C2 described below are used as the pretreatment solutions before oxidation dyeing.

After the treatment (i.e. the pretreatment before oxidation dyeing), each lock is rinsed with water and a new series of measurements is then performed. Thus, each series of measurements before and after treatment makes it possible to evaluate the variation in the stiffness K of the lock before and after the pretreatment before dyeing. The values are expressed as percentages of the value before treatment.

| | Var. Kdry | Var. Kwater | Var. ΔK |
|---|---|---|---|
| Product | (%) | (%) | (%) |
| Placebo | −32 ± 25 | −38 ± 21 | 2 ± 4 |
| (composition | | | |
| without | | | |
| active agent | | | |
| Polytetramethyleneguanidinium | −6 ± 10 | −15 ± 11 | 3 ± 4 |
| chloride | | | |

Var. Kdry corresponds to the variation in the stiffness in air of the hair (Kdry).

III) Var. Kwater corresponds to the variation of the stiffness of the hair, measured in water.

IV) Var. ΔK corresponds to the amplitude of the drop in the stiffness modulus during the immersion.

It emerges from the results of these measurements that polytetramethyleneguanidinium chloride has degradation-limiting effects.

It emerges from these tests that polytetra-methyleneguanidinium chloride has a protective effect on the keratin fibers of sensitized, dyed and permanent-waved hair when they are subjected to a pretreatment before oxidation dyeing.

III. Cosmetic Compositions

Compositions for Pretreatment Before Oxidation Dyeing

C1: Composition containing polytetramethylene-guanidinium chloride at pH 9.5

| | | |
|---|---|---|
| 50/50 C₂/C₁₀ alkyl polyglucoside as | 12 | g |
| a buffered aqueous 60% solution | | |
| polytetramethyleneguanidinium | 3 | g |
| chloride | | |
| pure absolute ethanol | 20 | g |
| pure benzyl alcohol | 4 | g |
| Polyethylene glycol (80 E) 400 | 6 | g |
| demineralized water | qs 100 | g |

C2: Composition containing polytetramethylene-guanidinium chloride at pH 7

| | | |
|---|---|---|
| polytetramethyleneguanidinium | 3 | g |
| chloride | | |
| pure absolute ethanol | 20 | g |
| diethylenetriaminepentaacetic | 2.4 | g |
| acid, pentasodium salt, as an | | |
| aqueous 40% solution | | |
| sodium metasulfite | 0.46 | g |
| demineralized water | qs 100 | g |

### Examples of Shampoo and Conditioners

| | | Shampoo | Conditioner |
|---|---|---|---|
| Composition | Shampoo 1 | 2 | 1 |
| Sodium lauryl ether | 12.5% AM | 15.5% | |
| sulfate as an aqueous | | AM | |
| solution containng 70% AM | | | |
| Cocobetaine as an | 2.5% AM | 2.4% | |
| aqueous solution | | AM | |
| containing 30% AM | | | |
| Cocamide MIPA (Empilan | | 0.5 | |
| CIS from Huntsman) | | | |
| Cetyl alcohol and | | 2.5 | |
| hydroxystearyl cetyl | | | |
| ether (Mexanyl GY from | | | |
| Chimex) | | | |
| Cetyl alcohol | | | 2 |
| Cetyl esters (Plant | | | 0.25 |
| Spermwax from Robeco) | | | |
| Behenyltrimethylammonium | | | 1.4% AM |
| chloride | | | |
| Lauryl PEG/PPG-18/18 | | | 0.15 |
| Methicone at 91% AM in | | | |
| isostearyl alcohol (Q2- | | | |
| 5200 from Dow Corning) | | | |
| Amodimethicone as a | | | 2.7 |
| cationic aqueous | | | |
| emulsion in 35% AM (DC | | | |
| 939 —Dow Corning) | | | |
| Dimethicone (Belsil | | 2.7 | |
| 300000 from Wacker) | | | |
| Polytetramethyleneguanidinium | 1% AM | | |
| oleate | | | |
| Polyethyleneguanidinium | | 0.5% | |
| oleate | | AM | |
| Polyethyleneguanidinium | | | 0.5% AM |
| chloride | | | |
| Carbomer (Carbopol 980 | | 0.2 | |
| from Noveon) | | | |
| Preserving agent | qs | qs | Qs |
| pH agent | qs pH = 7 | qs pH-7 | qs pH = |
| | | | 3.5 |
| Water | qs 100 | qs 100 | qs 100 |

1. A cosmetic composition, at least one compound of formula (I) or a compound derived therefrom: 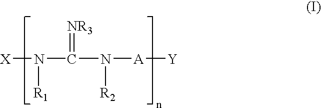
in which:
X, R₁, R₂ and R₃, independently of each other, are a hydrogen atom, a hydroxyl radical or a linear or branched, saturated or unsaturated, optionally hydroxylated C₁ to C₁₆ alkyl radical,
Y is a radical NHR, in which R is hydrogen atom, a hydroxyl radical or a linear or branched, saturated or unsaturated, optionally hydroxylated C₁ to C₁₆ alkyl radical,
A is a linear or branched, saturated or unsaturated, C₁ to C₁₆ alkylene radical optionally substituted with at least one of a hydroxyl, carboxyl radical, carboxylate radical or a halogen, wherein said alkylene radical may contain at least one —NH—, —O—, —S—, —(CO)O, of O(CO)—, —CONR—, NRCO—, in which R is hydrogen or a C₁ to C₈ alkyl radical, —NH(CO)O—, —O(CO)NH, —NH(CO)NH— function, a C₆ aryl or C₃ to C8 cyclanyl ring optionally substituted with a C₁-C₈ alkyl, hydroxyl or halogen; or A is one or more C₆ or C₇ aryl or C₅ to C₇ cyclanyl rings substituted with a C₁-C₈ alkyl, a hydroxyl group or a halogen or are unsubstituted; or A is a C₁₀ to C₁₄ polyaryl or C₆ to C₁₀ polycyclanyl chain, which may be interrupted with at least one C₁₀ to C₈ alkylene radical, an —NH—, —CONR—, or NRCO in which R is a C₁ to C₈ alkyl radical, —O—, —S—, a hydrogen atom, —NH(CO)O—, —O(CO)NH— or —NH(CO)NH— function;
n is an integer between 2 and 5,000 and, and the physiologically acceptable salts thereof.

2. The composition as claimed in claim 1, wherein the compound of formula I is at least one selected from the group consisting of polyethyleneguanidinium oleate, polyethyleneguanidinium chloride, polytetramethyleneguanidinium oleate, polytetramethyleneguanidinium acetate and polytetramethyleneguanidinium chloride.

3. The composition as claimed in claim 2, wherein the compound of formula I is polyethyleneguanidinium oleate.

4. The composition as claimed in claim 1, wherein the compound of formula (I) or a compound derived therefrom is present in an amount of from 0.001% to 25% by weight relative to the total weight of the cosmetic composition.

5. (Canceled).

6. (Canceled).

7. (Canceled).

8. (Canceled).

9. A compound derived from a polyguanidine of formula II: 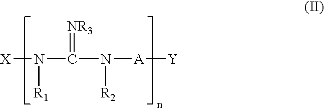
in which:
X, R₁, R₂ and R₃, independently of each other, are a hydrogen atom, a hydroxyl radical or a linear or branched, saturated or unsaturated, optionally hydroxylated C₁ to C₁₆ alkyl radical,
Y is a radical NHR, in which R is a hydrogen atom, a hydroxyl radical or a linear or branched, saturated or unsaturated, optionally hydroxylated C₁ to C₁₆ radical,
A is a linear or branched, saturated or unsaturated, C₁ to C₁₆ alkylene radical optionally substituted with at least one of a hydroxyl, carboxyl radical, carboxylate radical or a halogen, wherein said alkylene radical may contain at least one of an —NH—, —O—, —S—, —(CO)O—, O(CO)—, —CONR—, —NRCO— in which R is hydrogen or a C₁ to C₈ alkyl radical, —NH(CO)O—, —O(CO)NH—, —NH(CO)NH function, or a C₆ aryl or C₃ to C₈ cyclanyl ring optionally substituted with a C₁-C₈ alkyl, hydroxyl or halogen; or A is one or more of a C₆ or C₇ aryl or C₅ to C₇ cyclanyl rings on the condition that A is an unsubstituted linear C₁-C₁₆ alkylene radical, substituted with a C₁-C₈ alkyl, a hydroxyl group or a halogen or are unsubstituted; or A is a C₁₀ to C₁₄ polyaryl or C₆ to C₁₀ polycyclanyl chain, which may be interrupted with at least one C₁ to C₈ alkylene radical, an —NH—, —CONR—, NRCO in which R is a C₁ to C₈ alkyl radical, —O—, —S—, a hydrogen atom, carbamate —NH(CO)O—, —O(CO)NH— or —NH(CO)NH— function;
n is an integer between 2 and 5 000, and physiologically acceptable salts thereof.

10. The compound as claimed in claim 9, wherein A is a linear or branched, saturated or unsaturated C₁ to C₁₂ alkylene radical, substituted with at least one of a hydroxyl, carboxyl radical, carboxylate radical or a halogen.

11. A process for preparing a compound as claimed in claim 9, comprising
preparing a mixture comprising an approximately equimolar mixture of an alkylenediamine and a guanidine salt and heating this mixture to a temperature of between 120° C. and 150° C. for a period of between 4 and 10 hours.

12. The process as claimed in claim 11, wherein the mixture is prepared by mixing in bulk or in a solvent.

13. The process as claimed in claim 11, wherein or the heating is performed in two stages: heating to a temperature of between 80° C. and 120° C. for a period of between 2 and 5 hours, followed by heating to a temperature of between 120° C. and 150° C. for a period of between 5 and 11 hours.

14. A cosmetic composition comprising, in a cosmetically acceptable medium, at least one compound derived from the polyguanidine of formula (II) as claimed in claim 9.

15. The composition as claimed in claim 14, wherein the compound derived from the polyguanidine of formula (II) is present in an amount of from 0.001% to 25% by weight relative to the total weight of the composition.

16. The composition as claimed in claim 14, further comprising at least one adjuvant selected from the group consisting of fragrances, preserving agents, sequestering agents, thickeners and emulsifiers.

17. A cosmetic process for protecting, caring for and/or conditioning keratin fibers, comprising
applying to the keratin fibers at least one compound derived from the polyguanidine of formula (I): 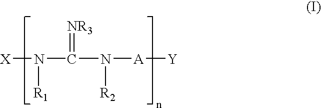
in which:
X, R₁, R₂ and R₃, independently of each other, are a hydrogen atom, a hydroxyl radical or a linear or branched, saturated or unsaturated, optionally hydroxylated C₁ to C₁₆ alkyl radical,
Y is a radical NHR, in which R is a hydrogen atom, a hydroxyl radical or a linear or branched, saturated or unsaturated, optionally hydroxylated C₁ to C₁₆ alkyl radical,
A is a linear or branched, saturated or unsaturated, C₁ to C₁₆ alkylene radical optionally substituted with at least one of a hydroxyl, carboxyl radical, carboxylate radical or a halogen wherein, said alkylene radical may contain at least one —NH—, —O—, —S—, —(CO)O—, O(CO)—, —CONR—, —NRCO— in which R is hydrogen or a C₁ to C₈ alkyl radical, —NH(CO)O—, or —O(CO)NH— —NH(CO)NH— function, a C₆ aryl or C₃ to C₈ cyclanyl ring optionally substituted with a C₁-C₈ alkyl, hydroxyl or halogen; or A is one or more C₆ or C₇ aryl or C₅ to C₇ cyclanyl rings substituted with a C₁-C₈ alkyl, a hydroxyl group or a halogen or are unsubstituted; or A is a C₁₀ to C₁₄ polyaryl or C₆ to C₁₀ polycyclanyl chain, which may be interrupted with at least one C₁ to C₈ alkylene radical, an —NH—, —CONR—, NRCO in which R is a C₁ to C₈ alkyl radical, —O—, —S—, a hydrogen atom, —NH(CO)O—, —O(CO)NH— or —NH(CO)NH— function;
n is an integer between 2 and 5 000, and the physiologically acceptable salts thereof.

18. The cosmetic composition as claimed in claim 1, wherein at least one of:
X, R₁, R₂ or R₃ is a linear or branched, saturated or unsaturated C₁-C₈ alkyl radical;
Y is a radical NHR in which R is a linear or branched, saturated or unsaturated C₁-C₈ alkyl radical;
A is a linear or branched, saturated or unsaturated C₁-C₁₂ alkylene radical; or
n is an integer of between 2 and 200.

19. The composition as claimed in claim 1, wherein the compound of formula (I) or the compound derived therefrom is present in an amount of from 0.1% to 10% by weight relative to the total weight of the cosmetic composition.

20. A shampoo comprising the composition as claimed in claim 1.

21. A conditioner comprising the composition as claimed in claim 1.

22. A keratin conditioner comprising the composition as claimed in claim 1.

23. The compound claimed in claim 9, wherein at least one of:
X, R₁, R₂ or R₃ is a linear or branched, saturated or unsaturated C₁-C₈ alkyl radical;
Y is a radical NHR in which R is a linear or branched, saturated or unsaturated C₁-C₈ alkyl radical;
A is a linear or branched, saturated or unsaturated C₁-C₁₂ alkylene radical; or
n is an integer of between 2 and 200.

24. The process as claimed in claim 11, wherein the mixture is prepared by mixing in polyethylene glycol.

25. A cosmetic composition comprising, in a cosmetically acceptable medium, at least one compound derived from the polyguanidine of formula (II) obtained by the process as claimed in claim 11.

26. The composition as claimed in claim 14, wherein the compound derived from the polyguanidine of formula (II) is present in an amount of from 0.1% to 10% by weight relative to the total weight of the composition.

27. The cosmetic composition as claimed in claim 17, wherein at least one of:
X, R₁, R₂ or R₃ is a linear or branched, saturated or unsaturated C₁-C₈ alkyl radical;
Y is a radical NHR in which R is a linear or branched, saturated or unsaturated C₁-C₈ alkyl radical;
A is a linear or branched, saturated or unsaturated C₁-C₁₂ alkylene radical; or
n is an integer of between 2 and 200.

28. A process comprising:
oxidation dyeing a substrate
and pretreating the substrate with the composition as claimed in claim 1 is present in pretreating the substrate.
